(19) United States
(12) Patent Application Publication
(10) Pub. No.: US 2005/0148886 A1
(43) Pub. Date: Jul. 7, 2005

(54) Digital indicator that can record human's wrist radial artery blood vessel pulsing information

(76) Inventors: Jiao Gong, Arcadia, CA (US); Wei Jiang, Arcadia, CA (US)

Correspondence Address: JIAO GONG, 411 S. 3RD AVE #B, ARCADIA, CA, 91006, US

(21) Appl. No.: 10/746,365

(22) Filed: Dec. 29, 2003

(51) Int. Cl. A61B 5/02

(52) U.S. Cl. 600/503; 600/504

(57) ABSTRACT

A digital indicator that can record human's wrist radial artery blood vessel pulsing information, consists of tentacle, pressure-adding device, pressure meter, controlling device, change alter, storing device, digital transition storing device, input connection device, battery and circuit board device. The invention uses wrist radial artery digital indicator, after collecting 18 kinds of artery blood vessel pulse standard value, graphs on radial artery, edits them into standard pattern data base software, provides the user that after the user input the information from the digital indicator, the determined input information for consulting results will be the output, decide the diagnostic of re-segmenting type of specific disease and disease group of 18 kinds of radial artery blood vessel pulse standard value, the difference between the determined value and the standard value could reason out the specific diagnosing result. It is a kind of innovation invention for American clinical medicine science.

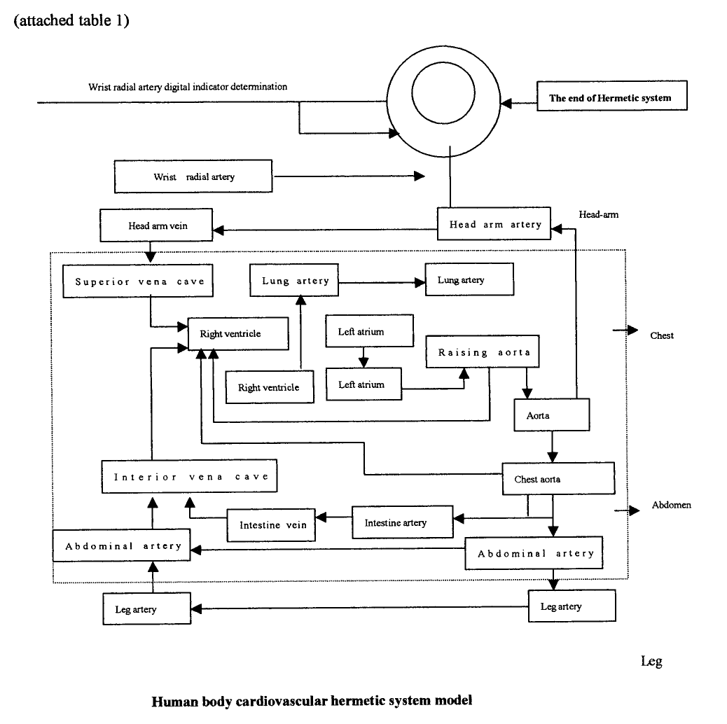

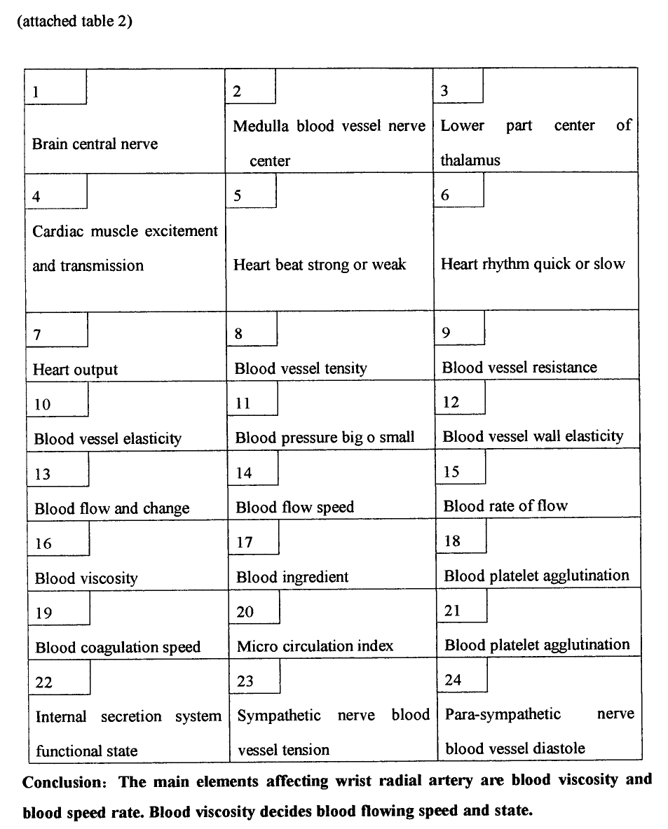

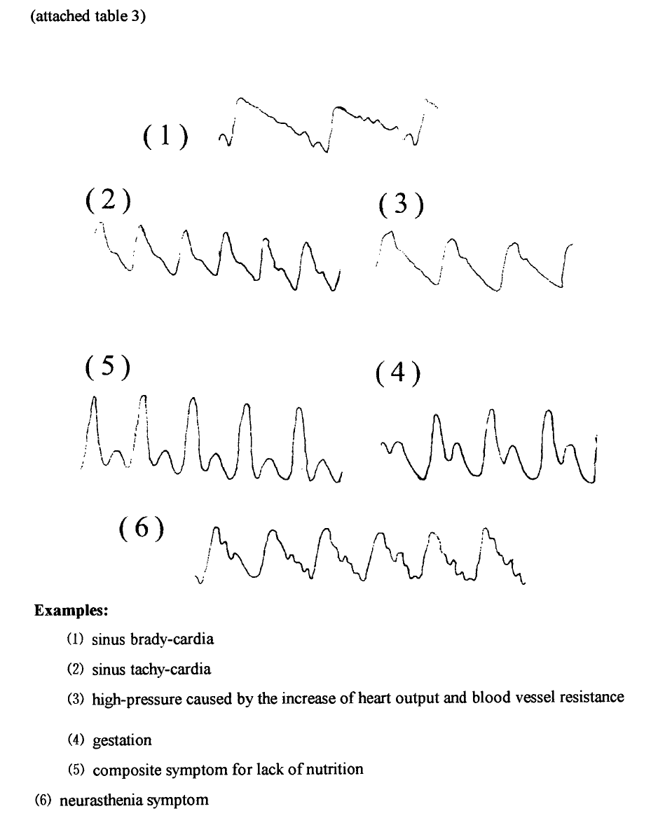

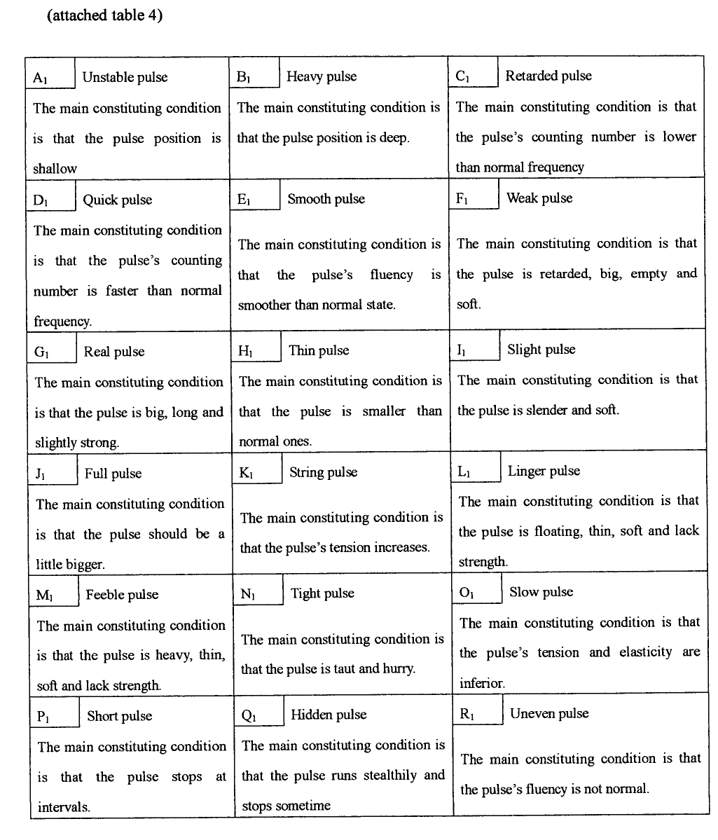

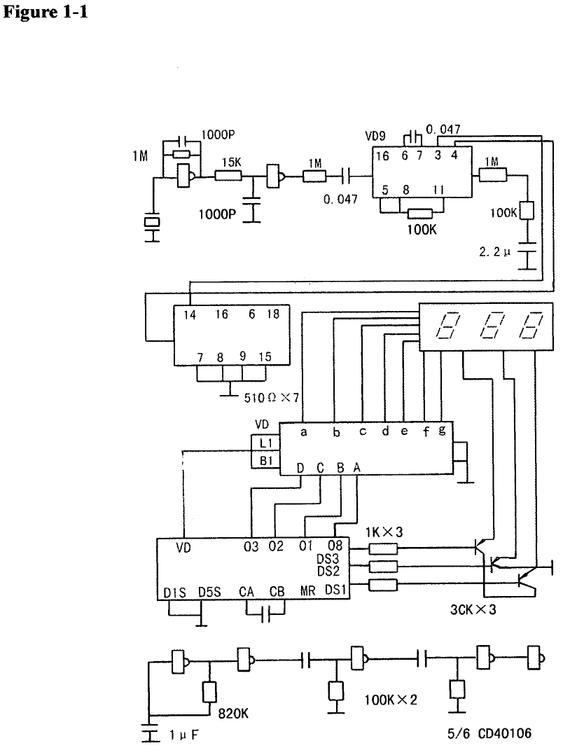

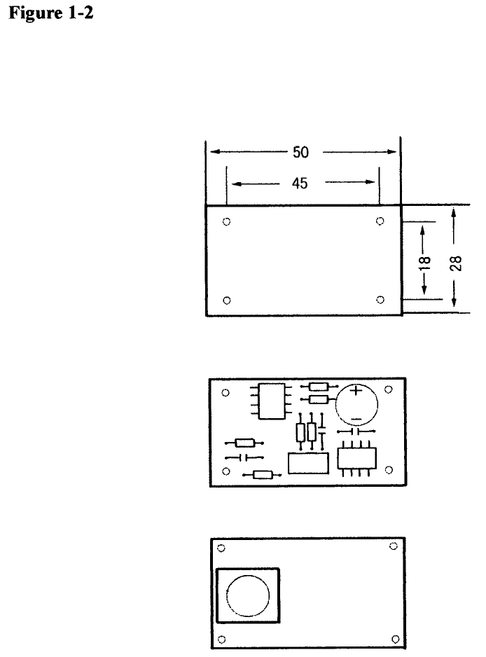

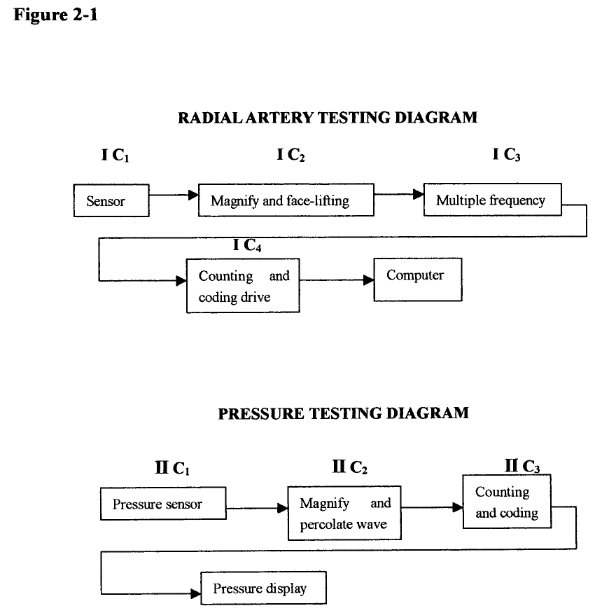

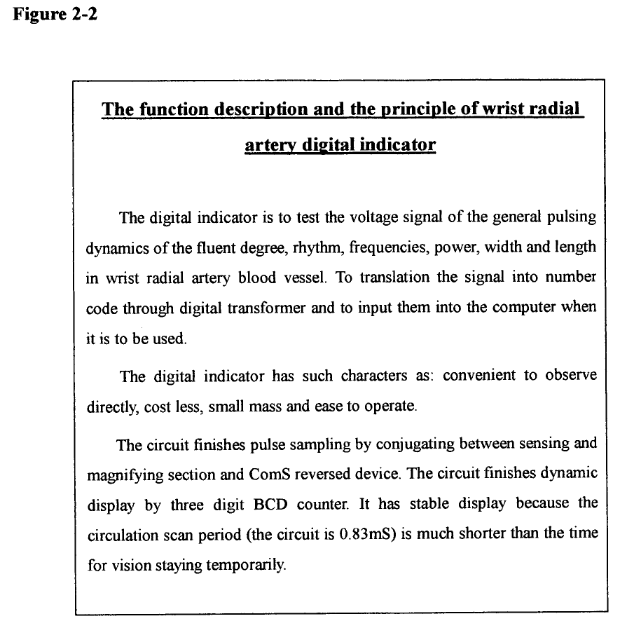

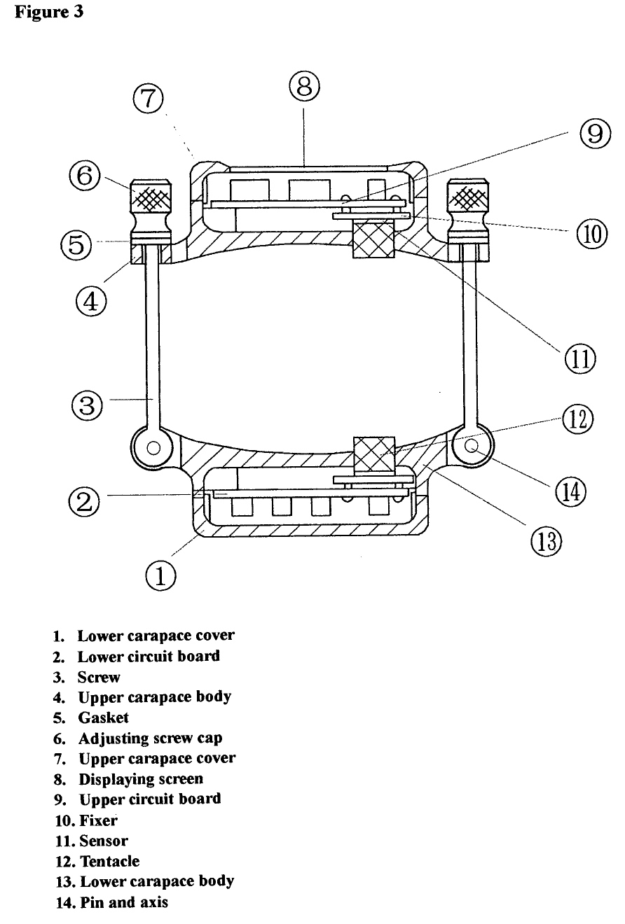

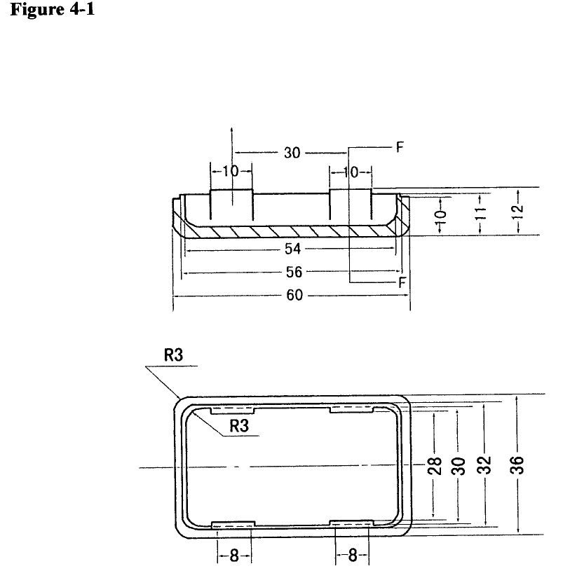

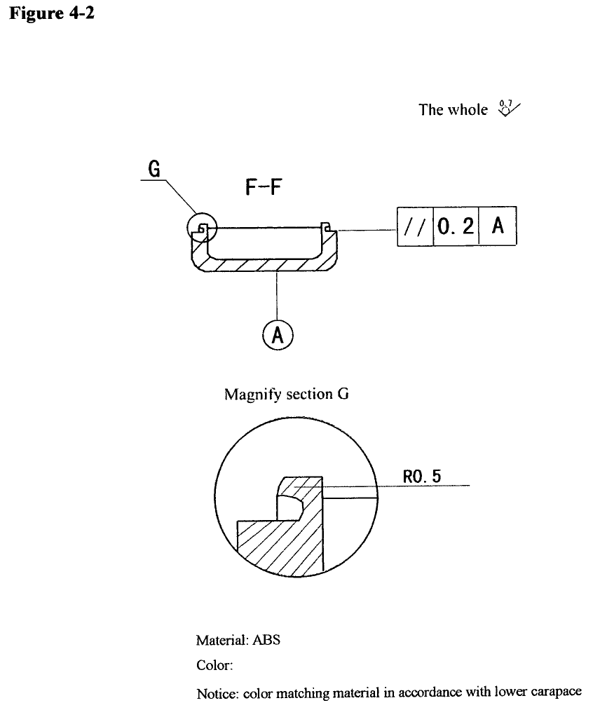

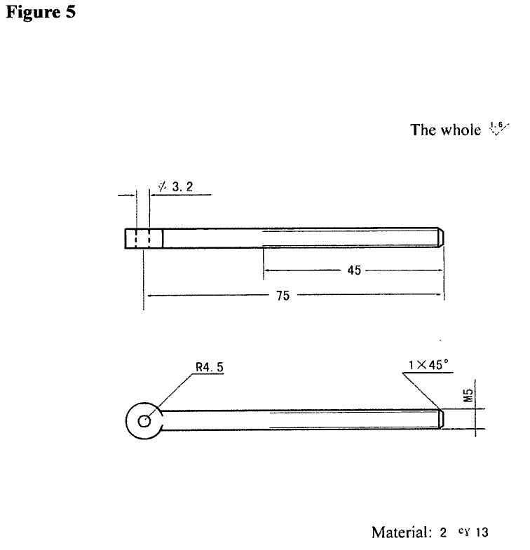

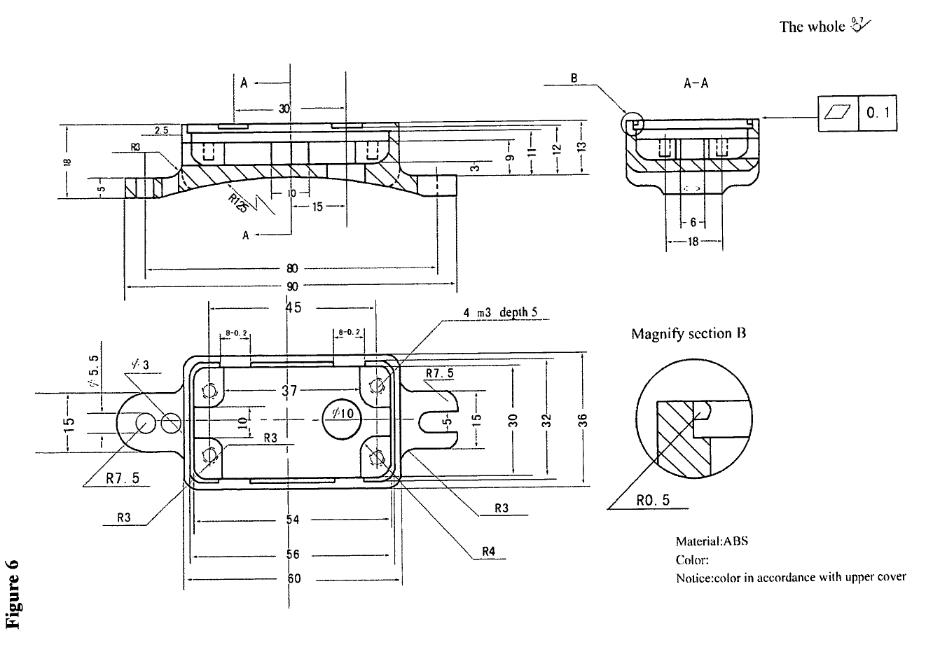

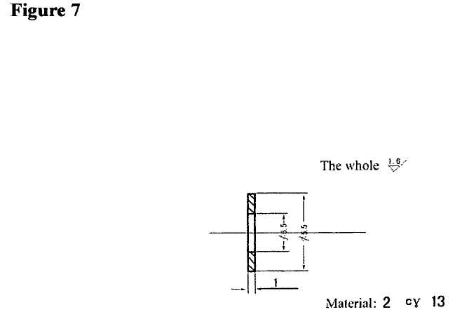

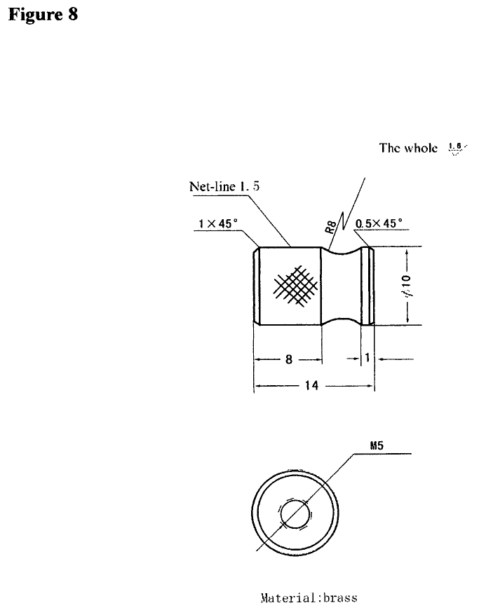

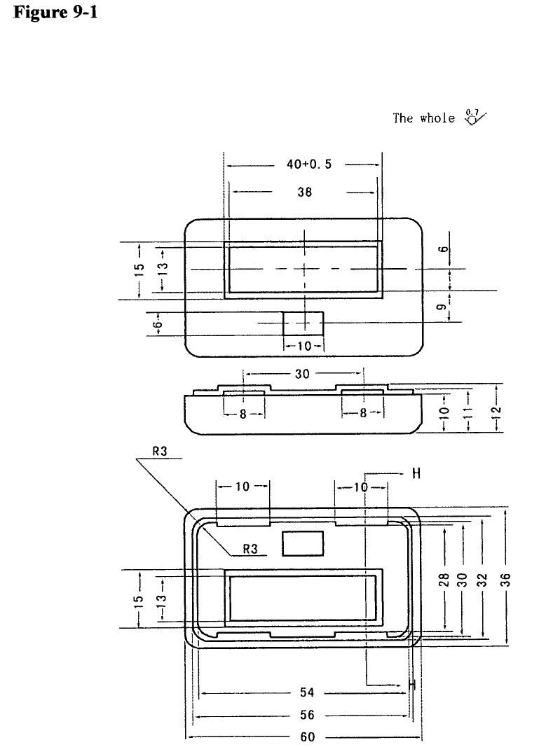

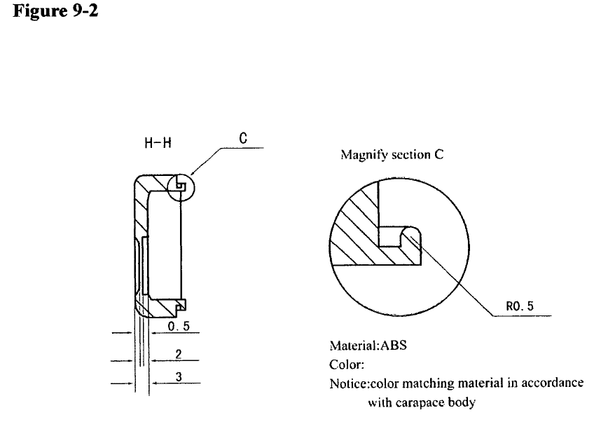

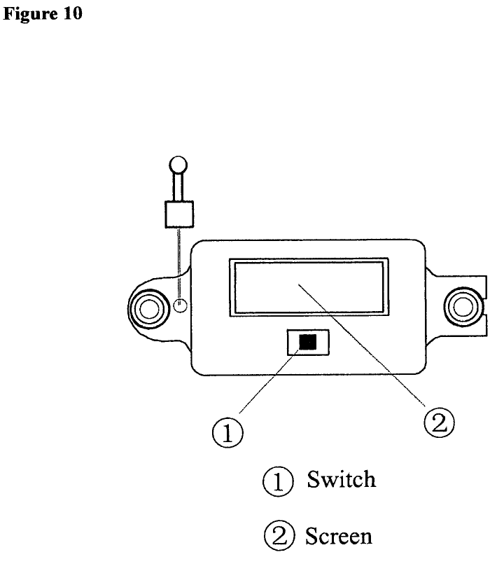

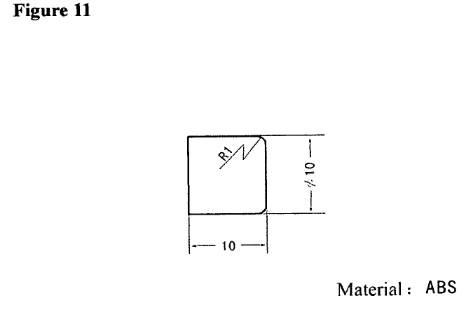

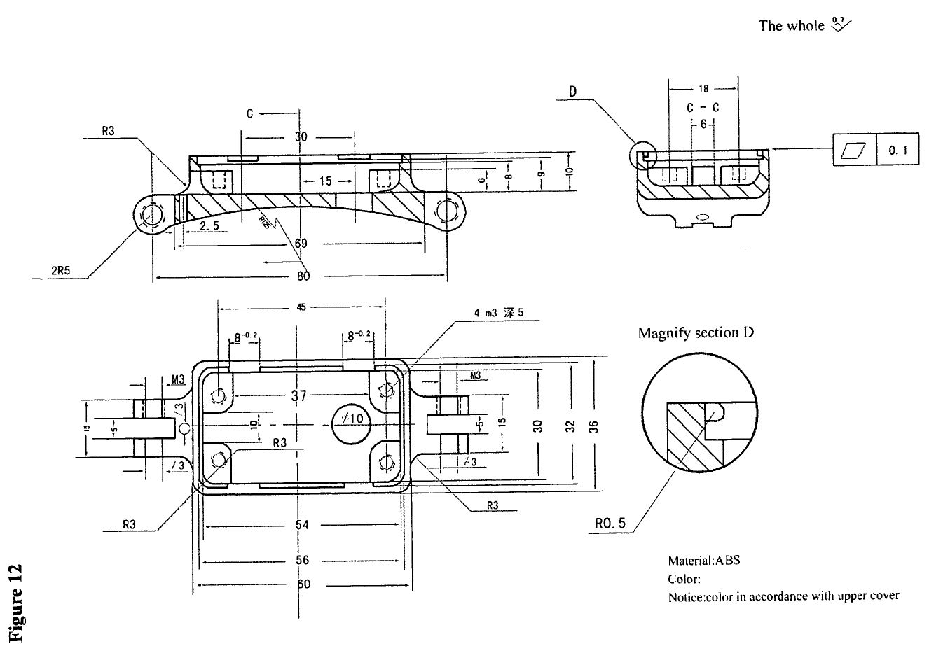

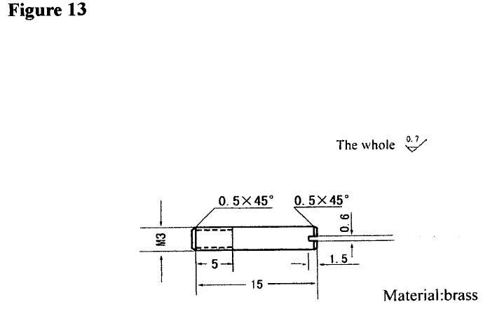

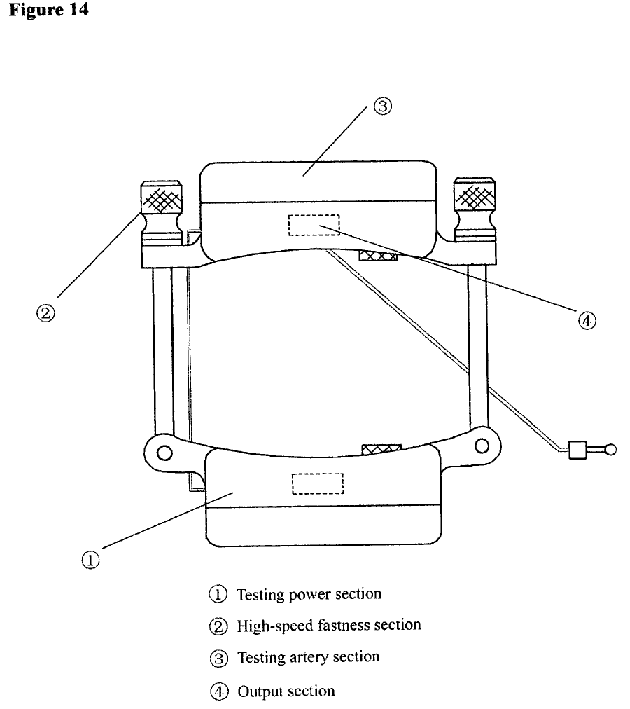

### BACKGROUND OF THE INVENTION

A digital indicator that can record human's wrist radial artery blood vessel pulsing information belongs to the invention patent of industrial manufacturing medical apparatus in American patent. The invention creates wrist radial artery digital indicator that can be used to collect and record the wrist radial artery blood vessel pulsing information to reflect the circulation, metabolism and tissue function status of all parts and systems in human body. And the digital indicator is used to undertake lots of health information checkup and technical examination and test to edit the information into logical and linear 18 standard value pictures, and store the information into the software of data base. Comparing the patient's radial artery blood vessel pulsing information tested by the digital indicator with the standard value pictures and data stored in the software of data base to analyze the difference, use the compared difference as the specific reasoning medical treatment diagnosis results of diagnosing disease and re-segmentation type through wrist radial artery blood vessel information, and can get the specific clue of medical treatment and specific diagnosis and treatment plan. It really reflects the patient's morbid status. It has realized intellectually and individually healthy medical treatment diagnosis and curing service.

In United States of America, the provenance of practicability term of patent may directly trace back to the phrases of patent clause in the Constitution that impower the congress to “make scientific and practical technology progress”. The term had been prescribed in the law of patent in 1790, 1836 and 1952.

No. 101 clause in American Patent Law stipulates that anyone who invents or discovers any novel and practical method, machine, product or compound, or their novel and practical improvement may gain patent according to its requirement and condition. Industrial practicability, manufacture or use in domain, which is also called manufacture or use in industry. Practicability only requires the technology plan of applying patent able to be manufactured or used in industry. If only there is the possibility able to be manufactured or used. It is not required to have been manufactured or used. In the patent law system of applying in advance, it is undoubtedly more practiced to apply first and then to manufacture or use in industry. If it is really manufactured or used in industry first and then applied for patent, that would lead its novelty lost.

In USA novelty requires to be embodied in the definition of may-obtain patent per se, the invention that can win patent is not only original but also initial. In order to determine the finished time and the priority of invention, American Patent & Trademark Bureau and Federation Court have outcome to create the concepts of Conception, Reduce to practice and Diligence. Conception is the spirit activities of invention, Reduce to practice may be the real one or putative one. Putative Reduce to practice means putting in the patent application plan for making inventing new thought to the view and the method to complete the new thought. Diligence is the rational endeavor made by the inventor to realize his invention conception. The invention creativity has been defined with non-obviousness in the law of American patent. The stipulation of non-obviousness of invention theme appeared in No. 103 clause of the patent law in 1952 for the first time. The examination guide of American Patent & Trademark Bureau stipulates weather the invention applying for patent, as a whole, is non-obviousness to the time and the person mentioned in the formed part. A digital indicator that can record human's wrist radial artery blood vessel pulsing information has answered for and satisfied with the patentable practicability term, the novelty standard and the creativity requirement of American Patent & Trademark Bureau.

Both the digital indicator that can record human's wrist radial artery blood vessel pulsing information and the “network hospital of diagnosing disease and re-segmentation type with the wrist radial artery blood vessel pulsing information” method applied by me, belong to the same creative system, a kind of new medicine diagnosing method that contains less present technologies in contrast with those applied in American medical treatment circle. In clinical treatment, there are always some patients who fell flustered, stuffy, palpitated, pant, there are also some other patients who often have diagnostic of getting angry, but the results show that they are normal after the examinations by all kinds medical instruments such as electro cardiogram. B-ultrasonic, CT, chemical examination and coronary arteries (veins) radiography American medicine must also complete the transition from collecting macro-information to collecting micro-information. Information message and news, are also signal and symbols. The pictures and digital information of wrist radial artery blood vessel pulse mainly reflect the physiologic and pathologic phenomenon in cardiovascular system, internal secretion and nervous system, they are quantitative and numerical information. Reflecting the organic physiologic and pathologic changes through radius artery, collecting those information with rich content and integration by wrist artery blood vessel digital indicator, contrasting the differences within the testing value and the standard value, provide the specific disease re-segment diagnosis and treating plans.

A digital indicator that can record human's wrist radial artery blood vessel pulsing information, compared with digital blood-pressure-meter, the digital indicator may obtain more changing information of organic physiology and pathology and medicine diagnosis basis by analyzing and determining the curves, standard curves and the pulsing information curves of wrist radial artery blood vessel. Digital blood-pressure-meter only simply records the maximum and minimum pressure within the blood vessel of human's body.

### BRIEF SUMMARY OF THE INVENTION

Human body is an organic, comparative closed circulatory. Wrist radius artery is the end of this comparative closing whole, is perfect organic information recourses, and also a collecting point for the whole information. This patent uses wrist radius artery digital indicator to input the accurately obtained data information at the end of radius artery and out put the consulting results of the tested value information, realizes storing and dealing with those data, and answer the inquires from customer end, operates continually.

The signals reflected by wrist radius artery blood vessel pulse show the relationship among the blood circulatory; plant nerve and internal secretion system, which have their own information system to handle their own information. Because all the organs, tissues in the organic have their method to gain, utilize, keep and transmit the information. In normal condition, information transmission among the different parts has enough reliability, the adjustment and control information among them all could be received by relative part and then makes reaction. The artery blood pressure adjustment of a normal person is also a kind of reaction adjustment. Organic tissues all have their own information system, which gather into central nerve, the adjust center of the central nerve reacts, so, all the information system have the function to use, keep and transmit the reacting information which unite all the parts of organic whole together to form a whole to keep organic stable. The whole set of information organic state reflects in the database through its important end, wrist radial artery blood vessel pulse. The network hospital diagnosis method of diagnosing disease and re-segment type through wrist radial artery blood vessel pulsing information is just using wrist radius artery digital indicator to seek valuable end radial artery information for doctors to diagnose diseases and assistant to diagnose diseases. The digital indicator is a kind of innovation, a practical American new medical apparatus with novelty. The digital indicator consists of tentacle, pressure-adding device, pressure meter, controlling device, digital transition storing device, input connection device and battery. Its function is to test the voltage signal of the general pulsing dynamics of the fluent degree, rhythm, frequencies, power, width and length in wrist radial artery blood vessel. To translation the signal into number code through digital transformer and to input them into the computer when it is to be used. Animal experiments show that the radial artery blood vessel pulsing information and graph curves gained from internal radial artery by inserting into the artery vessel with wound and from the radial artery surface by using wrist radial artery digital indicator, the parameters for calculating blood flowing dynamics are basically the same, its relativity is good, it indicts that the industrial invention by using wrist radial artery digital indicator, studying on-wound blood flowing dynamics parameter and collecting the information of human body's state from the pulse of radial artery, has great future for its usage and development. Radial artery blood vessel carries a great deal of human body circulatory dynamics information that is precious wealth of human health invent by which is the final purpose to wrist radial artery digital indicator.

### BRIEF DESCRIPTION OF THE SEVERAL VIEWS OF THE DRAWING

Table of Contents

Table I The principle of testing wrist radial artery blood vessel pulse information by wrist radial artery digital indicator. (see attached table 1)

Table 2 The main elements affecting the graphs and results of wrist radial artery blood vessel pulse information tested by wrist radial artery digital indicator. (see attached table 2)

Table 3 The graphs of wrist radial artery blood vessel pulse information determined practically by wrist radial artery digital indicator and the standard graphs. (see attached table 3)

Table 4 The standard result menu of wrist radial artery blood vessel pulse tested by wrist radial artery digital indicator (see attached table 4)

### DETAILED DESCRIPTION OF THE INVENTION

1. The digital indicator that can record human body wrist radial artery consists of tentacle, pressure-adding device, pressure meter, controlling device, digital transition storing device, input connection device and battery. Its function is to test the voltage signal of the general pulsing dynamics of the fluent degree, rhythm, frequencies, power, width and length in wrist radial artery blood vessel. To translation the signal into number code through digital transformer and to input them into the computer when it is to be used. The way for using wrist radial artery digital indicator is: the examinee lies on the bed, clam and relaxed, breathing calmly upper limbs stretching, the center of the palm upward, leaves wrist exposed, to be touched with finger for the pulsing apparently place, placing the center of indicator tentacle film aiming at the artery vessel, press the indicator's wrist ring structure and fasten the indicator. Press down the pressure-adding function button, waiting for it reaching to 100 mm Hg till it is stable at the value, and the information of wrist radius artery blood vessel pulse is in stable state, the indicator lamp at the side of the indicator window gives off yellow lights, the indicator starts work automatically. After collecting 4 sets of ware pattern of radius artery blood vessel pulse information, to turn off the button with single tough. Animal experiments show that the radius artery blood vessel pulsing information and graph curves gained from internal radial artery by inserting into the artery vessel with wound and from the radius artery surface by using wrist radial artery digital indicator, the parameters for calculating blood flowing dynamics are basically the same, its relativity is good, it indicts that the study for on-wound blood flowing dynamics parameter and the diagnosis method for human body's state from the pulse of radial artery by using wrist radius artery digital indicator has great future for its usage of development. Radial artery blood vessel carries a great deal of human body circulatory dynamics information. Collecting and recoding these valuable information to serve Americans' health is the final purpose for inventing wrist radial artery digital indicator.

2. Human body is an organic whole, its blood circulatory systems are close series and mutual establish ties system, wrist radius artery is the end of the whole hermetic system (table 1), the patent is an innovation to the clinical medical system collecting radial artery blood vessel pulse information at the end of the circulation by using wrist radius artery digital indicator. Wrist radial artery blood vessel shows the function and affection of heart functional state, blood vessel functional state and blood's quality and quantity. It also reflects the functional state of plant nervous system and internal secretion system. Because of circulatory system, the affection could be read out indirectly in the radial artery pulse information. It also shows the functional states of organic plant nerve, internal secretion system and brain central system (Table 2). Collection and analysis of wrist radial artery blood vessel pulse information provide important illustrations for the characters of many kinds of disease.

3. The invention uses wrist radial artery digital indicator, with the assistance of professional acupuncturist, collects massively human body 18 kinds of standard radius artery blood vessel pulsing information pictures and standard data, organizes the data with the way of digital transition, completes logical system coding after building up immense data base software of radial artery blood vessel pulse information logical system, and then manufactures and sent freely a lot of wrist radial artery digital indicator to network hospital membership, starts the medical activities like health consultation, diagnosis and treatment, buying commodities with computers. Wrist radial artery is one of the most important, sensitive and reliable information resources of human activities. The information of radial artery pulse is a testable biological signal in the developing process of internal secretion system and nervous system within human circulatory system, the graphs formed with those human body valuable signal by recording according to its order with the same pressure are radial artery blood vessel pulsing graphs. With using wrist radial artery digital indicator, 30 professional acupuncturists who had passed the examination about professional practitioners for acupuncture and moxibustion major given by the government of USA, arrange 1000 volunteers in USA into many groups, there are three persons in one group, collect 18 kinds of typical, standard wrist radial artery blood vessel pulse information graphs and division, through the software edition process from editing input, effective data, linear program for solution, results patternized, handle the logic data system elements of determined value into logic system, finish the applying system from input of the gained data determinating value information to 18 kinds of standard, typical radial artery blood vessel pulse information standard value graphs and digital codes, which have been stored in the data base of website, server and computers.

4. A kind of digital indicator that can record wrist radial artery blood vessel pulse information, the decided 18 kinds of radial artery pulse information themselves can show the morbid state's difference (table 3). To identify the specific difference should compare the specific features of a disease or a group of disease with the standard value to point out the specific diagnostic of disease re-segment and the specific pathogens. For example: a patient has a cute pain in chest, with the diagnostic like cough, chest stuffy, night sweat, after gaining the wrist radial artery digital indicator and standard pattern data base software, through testing, the computer screen shows the patient's blood viscosity high and the type of figure extra fat, diagnosing that the patient has the disease of high pressure because of output and blood vessel resistance incensement. The database takes the determined value to compare with the standard value in the database, make sure its standard value. According to the main feature of artery blood vessel pulse tension increasing, re-segment it as blood viscosity high, pressure high, fatty, has the diagnostic of blood silted up.

1. I announce that “a digital indicator that can record human's wrist radial artery blood vessel pulsing information” is my invention.
I think my invention consists of tentacle, pressure-adding device, pressure meter, controlling device, change alter, storing device, digital transition storing device, input connection device, battery and circuit board device.
I think that my invention, the digital indicator, has the function to test the information of the general pulsing dynamics of the fluent degree, rhythm, frequencies, power, width and length in wrist radial artery blood vessel. To input them into the computer when it is used.
I think that my invention includes the principle of wrist radial artery blood vessel pulse information determined by wrist radial artery digital indicator, information graphs and the main elements for the results, standard graphs and standard result menu.
